(19) United States
(12) Patent Application Publication
(10) Pub. No.: US 2007/0258961 A1
(43) Pub. Date: Nov. 8, 2007

(54) Cholesterol efflux and uses thereof

(76) Inventors: Zygmunt Krozowski, Victoria (AU); Dmitri Sviridov, Victoria (AU)

Correspondence Address: Michael L Goldman;Nixon Peabody, Clinton Square, PO Box 31051, Rochester, NY, 14603, US

(21) Appl. No.: 10/529,096

(22) PCT Filed: Sep. 23, 2003
(86) PCT No.: PCT/AU03/01256

§ 371 Date: Oct. 17, 2005

(30) Foreign Application Priority Data
Sep. 24, 2002 (AU) 2002951611

(51) Int. Cl. A61K 48/00; A61K 39/00; C12N 5/06; C12Q 1/68

(52) U.S. Cl. 424/93.21; 435/325; 435/375; 435/6; 514/44

(57) ABSTRACT

In a first aspect of the present invention, there is provided a method of modulating cholesterol efflux in a cell, said method comprising modulating expression and/or activity of sterol 27-hydroxylase (CYP27) and/or caveolin-1 in the cell. Applicants have found that cholesterol can be removed by cholesterol efflux which is regulated by CYP27 and/or caveolin-1. More suprisingly for CYP27 modulation the cholesterol effluxed remains in the form of cholesterol and not as oxidized cholesterol as would be expected in the presence of CYP27. Preferably, CYP27 is modulated in macrophages and caveolin-1 is modulated in hepatic cells.

The present invention relates generally to modifying cell metabolism particularly, metabolism of cholesterol and to methods of modifying efflux of cholesterol from cells. The invention also relates to methods of preventing and treating cholesterol related conditions such as atherosclerosis by modifying cholesterol metabolism and efflux from cells which may reduce the incidence of atherosclerosis by preventing accumulation of cholesterol in cells and by enhancing conditions for cholesterol efflux.

### BACKGROUND

Atherosclerosis is a disease of the arteries characterised by the appearance of fatty lesions along the inner surface of a blood vessel wall, also known as atheromatous plaques. Contributing to the narrowing of the vessel lumen, the resultant constriction to smooth blood-flow ultimately deprives vital organs of their blood supply. Atheromatous plaques also have an increased propensity to rupture. Thrombi formed on the surface of ruptured plaque become dislodged from the vessel wall, thus contributing to further end-organ damage by becoming lodged in arteries supplying the heart, causing myocardial infarction (heart attack); or the brain, causing stroke. The clinical and social significance of this disease is evident in the fact that half the annual mortality in Western society results from heart and blood-vessel diseases of which atherosclerosis is the primary cause. Understanding the underlying causes of atherosclerosis is thus vital in the design of effective treatment strategies in combating this alarming statistic.

A key event in the formation of the earliest atherosclerotic lesion, the fatty streak, is the influx of macrophages into the artery wall and their subsequent uptake of abnormal lipoprotein particles and cholesterol, forming what are often referred to as foam cells. Cholesterol, being a major constituent of the atherosclerotic plaque, has thus become the major focus of treatment strategies.

Being the major sterol in the human body, cholesterol has, as its primary function, the structural integrity of cell membranes. It is also implicated in vital bodily functions, and synthesis of a number of steroid hormones, including estrogen, progesterone and testosterone. In the liver, cholesterol is also the precursor of the bile acids which, when secreted into the intestine, aid in digestive processes.

Whilst important to human physiology, elevated levels of cholesterol in the blood, and the type of lipoproteins that transport it through the blood, clearly promote the onset and progression of atherosclerosis. However, current treatment strategies designed to lower cholesterol levels in the blood and in the cells, whether it be by dietary means or via lipid-lowering drugs, have been shown to slow or even halt progression of atherosclerosis and to reduce the risk of end-organ damage, but have had no impact on the regression of existing atherosclerotic plaques.

The limitations in presently available treatments as they relate to cholesterol-lowering therapies, highlight the need for novel therapeutic approaches in attenuating the progression, and ultimately in promoting the regression or preventing the establishment, of atherosclerotic lesions. One such approach is the elimination of cholesterol from foam cells, stabilizing them against lethal rupture, and more importantly, promoting the regression of existing lesions and a reversal of disease severity. The elimination of cholesterol from macrophage foam cells of atherosclerotic plaques, or cholesterol efflux, appears to be regulated by a number of factors, both intracellular and extracellular.

Accumulation of cholesterol is a result of an imbalance between pathways delivering cholesterol to cells and removing it. Pathways responsible for delivery of cholesterol to cells have been investigated and treatment based on this knowledge has been applied with great success. To achieve further progress in reducing atherosclerosis and consequently the risk of heart diseases and stroke, the pathway related to removing cholesterol from cells may also be targeted. This pathway is the reverse cholesterol transport (RCT) pathway.

The RCT pathway removes excess cholesterol from extrahepatic tissues including the vessel wall, thus preventing development of atherosclerosis. On the other hand RCT is a major source of HDL, which has strong anti-atherogenic properties either through its role in RCT or by other mechanisms. The first and most likely rate-limiting step of reverse cholesterol transport is cholesterol efflux which is the transfer of cholesterol from cells to acceptors in plasma. Two pathways of cholesterol efflux are currently known. One involves lipidation of lipid-free or lipid-poor apolipoprotein A-I (apoA-I), and is most likely mediated by the ABCA1 transporter. The other involves transfer of cholesterol from plasma membrane caveolae to lipidated apoA-I or mature high density lipoprotein (HDL). However, control of these mechanisms of efflux remains unclear. Enhancement of cholesterol efflux from the vessel wall would protect against development of atherosclerosis by lowering cholesterol content of macrophages in vessel wall. Enhancement of cholesterol efflux from liver would protect against development of atherosclerosis by raising HDL concentration in blood, which has multiple anti-atherogenic effects. These mechanisms alone or in combination may assist in the control of cholesterol levels in the body and the risk of cholesterol related conditions.

Cholesterol oxidation in the liver is a major pathway of cholesterol catabolism resulting in the conversion of cholesterol to bile acids. Oxidation of cholesterol in other cells such as macrophages and endothelial cells may also occur and is catalysed by the enzyme sterol-27 hydroxylase (CYP27) which converts cholesterol to 27 hydroxy cholesterol and 3-beta-hydroxy-5-cholestenoic acid. However, these oxidized forms of cholesterol may be toxic to the cells and are often released from the cells since the oxidized form of cholesterol is more hydrophilic. A build up of these byproducts is therefore undesirable for reasons of toxicity and deposition of these oxidized forms of cholesterol may lead to plaque formation, atherosclerosis and coronary heart disease. Whilst oxidation might be beneficial converting cholesterol to a form that can be easily released from the cell, the conversion must be regulated at a level that is non toxic or detrimental to the cell. Therefore, it would be desirable to increase cholesterol efflux without increasing toxic oxidized cholesterol byproducts.

It is an objective of the invention to improve cholesterol efflux, enhance protection against accumulation of cholesterol, development of atherosclerosis and heart disease.

### SUMMARY OF THE INVENTION

In a first aspect of the present invention, there is provided a method of modulating cholesterol efflux in a cell, said method comprising modulating expression and/or activity of sterol 27-hydroxylase (CYP27) and/or caveolin-1 in the cell.

Applicants have found that cholesterol can be removed by cholesterol efflux which is regulated by CYP27 and/or caveolin-1. More surprisingly for CYP27 modulation the cholesterol effluxed remains in the form of cholesterol and not as oxidized cholesterol as would be expected in the presence of CYP27.

In a further preferred aspect of the present invention, there is provided a method of modulating cholesterol efflux in a cell, said method comprising:
transfecting the cell with a gene encoding CYP27 and/or caveolin-1; and
modulating expression and/or activity of the transfected CYP27 and/or caveolin-1 in the cell.

This method of modulating cholesterol efflux by directly affecting expression of CYP27 and/or caveolin-1 is preferentially used to modulate CYP27 and/or caveolin-1 expression from the natural levels. Preferably, the introduction of a construct with the gene for CYP27 and/or caveolin-1 will increase the level of CYP27 and/or caveolin-1 upon expression and thereby affect the cholesterol efflux.

In a preferred aspect there is provided a method of increasing cholesterol efflux in a cell, said method comprising
increasing expression and/or activity of CYP27 and/or caveolin-1 in the cell.

In a further preferred aspect of the present invention, there is provided a method of increasing cholesterol efflux from a cell, said method comprising
transfecting the cell with a gene encoding CYP27 and/or caveolin-1; and
increasing expression of the transfected CYP27 and/or caveolin-1.

In another aspect of the present invention there is provided a cell with modulated cholesterol efflux, said cell having modulated CYP27 and/or caveolin-1 expression and/or activity.

Preferably the cell is transfected with a gene encoding CYP27 and/or caveolin-1. The cell is therefore capable of modulation, preferably having increased cholesterol efflux upon increased CYP27 and/or caveolin-1 expression.

In yet another aspect of the present invention, there is provided a method of treating a cholesterol related condition in a patient by modulating cholesterol efflux from a cell of the patient, said method comprising:
modulating expression and/or activity of CYP27 and/or caveolin-1 in the cell.

In a further preferred aspect of the present invention, there is provided a method of treating a cholesterol related condition in a patient by modulating cholesterol efflux in the patient, said method comprising:
introducing modulated cells to the patient, wherein said cells have modulated expression and/or activity of CYP27 and/or caveolin-1.

In yet another aspect of the present invention there is provided a method of identifying a compound which modulates cholesterol efflux in a cell, said method comprising:
contacting the compound to the cell;
detecting a change in CYP27 and/or caveolin-1 expression and/or activity in the cell relative to a cell which has not been contacted with the compound.

### FIGURES

FIG. 1 shows synthesis of 27-hydroxycholesterol in cells transfected with CYP27. CHOP cells were transfected with CYP27A1 or mock transfected with pcDNA1 for 48 h. Following transfection, cells were incubated for 24 h in serum-free medium containing [1-¹⁴C] acetate. Cells were then washed and incubated for 2 h with 5% human plasma. Sterols from both cells and medium were extracted and separated with TLC and counted.

FIG. 2 shows cholesterol efflux to human plasma and lipid-free apolipoprotein A-I. CHOP cells transfected with CYP27, mock-transfected and non-transfected were labeled with [³H] cholesterol. Cells were then incubated with human plasma (final concentration 5%), human lipid-free apoA-I (final concentration 30 μg/ml) or serum-free medium alone for 2 h at 37° C. in a CO₂-incubator. Medium was collected, cells washed and the amount of radioactivity in the cells and medium determined on a β-counter. Cholesterol efflux is expressed as the percentage of labeled cholesterol moved from cells to medium (i.e. radioactivity in the medium/radioactivity in the medium+radioactivity in the cells). Means ±SD of quadruplicate determinations are shown. *p<0.001 versus mock-transfected and nontransfected cells.

FIG. 3 shows time-course (A) and dose-dependence (B) of cholesterol efflux to human plasma. CHOP cells transfected with CYP27 or mock-transfected were labeled with [³H] cholesterol. Cells were then incubated with human plasma added at a final concentration of 5% (A) or at the indicated concentrations (B), or serum-free medium alone at the indicated periods of time (A) or for 2 h (B) at 37° C. in a CO₂-incubator. Medium was then collected, cells washed and the amount of radioactivity in the cells and medium determined in a β-counter. Cholesterol efflux is expressed as the percentage of labeled cholesterol moved from cells to medium (i.e. radioactivity in the medium/radioactivity in the medium+radioactivity in the cells). Background values (i.e. the efflux to the medium alone) were subtracted. Means ±SD of triplicate determinations are shown.

FIG. 4 shows expression of caveolin-1 mRNA (A) and protein (B) in HepG2 and GepG2/cav cells. (A.) Expression of caveolin-1 mRNA was analyzed by RT-PCR as described. The expected size of the amplicon is 394 base pairs. (B). Western blot of transfected and non-transfected cells using anti-caveolin-1 antibody. Line 1—endothelial cells; Line 2 & 3—different amounts of HepG2/cav cells; Line 4—HepG2 cells.

FIG. 5 shows ultrastructural analysis of caveolae in transfected HepG2 cells (A) and analysis of transfected HepG2 cells with confocal microscopy. (A)—Cells were processed for embedding in epon and sections were cut parallel to the substratum. Uncoated flask-shaped pits or vesicular profiles typical of caveolae are evident in the transfected cells (arrowheads; contrast with larger clathrin coated pits indicated by arrows). Bars, 100 nm. (B)—Cells were fixed in acetone for 20 min washed with PBS and incubated for 1 h with anti-caveolin-1 antibody, washed and incubated in the dark for 1 h with Texas Red labeled anti rabbit IgG.

FIG. 6 shows cholesterol efflux to human plasma and lipid-free apolipoprotein A-I. Transfected and non-transfected cells were labelled with [³H]cholesterol (A) or [³H]acetate (B) as described. Cells were then incubated with the indicated concentrations of human plasma or human lipid-free apoA-I (final concentration 50 μg/ml) or cyclodextrin (final concentration 200 μg/ml) or serum-free medium alone for 2 h at 37° C. in a CO₂-incubator. (A)—Medium was collected, cells washed and the amount of radioactivity in the cells and medium determined in by liquid scintillation spectrometry. (B)—cholesterol was isolated from cells and medium by TLC as described. Cholesterol efflux is expressed as the percentage of labelled cholesterol moved from cells to medium (i.e. radioactivity in the medium/radioactivity in the medium+radioactivity in the cells). Means ±SD of quadruplicate determinations are shown. (*p<0.001 versus non-transfected cells).

FIG. 7 shows phospholipid efflux to lipid-free apolipoprotein A-I. Transfected and non-transfected cells were labelled with [³H] choline as described. Cells were then incubated with human lipid-free apoA-I (final concentration 50 μg/ml) for 2 h at 37° C. in a CO₂-incubator. Phospholipids were isolated from cells and medium by TLC as described. Phospholipid efflux is expressed as the percentage of labelled phospholipid moved from cells to medium (i.e. radioactivity in the medium/radioactivity in the medium+radioactivity in the cells). Means ±SD of quadruplicate determinations are shown. (*p<0.05 versus non-transfected cells).

FIG. 8 shows evaluation of the amount of cholesterol in cholesterol-rich domains (A) and rate of cholesterol trafficking from endoplasmic reticulum to cholesterol-rich domains. The entire cellular cholesterol pool was labelled by incubating in a serum-containing medium with [³H] cholesterol for 48 h at 37° C. in a CO₂ incubator. After washing cells were further incubated in serum-free medium for 18 h at 37° C. in a CO₂ incubator. Cells were cooled on ice, [¹⁴C] acetate was added and cells were further incubated for 3 h at 15° C. At the end of the incubation cells were quickly warmed and incubated for 20 min at 37° C. to allow a portion of the newly synthesized cholesterol to be transferred to the plasma membrane. The cells were cooled on ice, washed 3 times with the ice-cold phosphate buffered saline (PBS). Cholesterol oxidase (Roche) was added to the cells at a final concentration of 1 unit/ml and flasks were incubated for 3 h at 4° C. Lipids were extracted from cells and analyzed by TLC as described in “Materials and Methods”. The amount of oxidazable cholesterol was calculated as the amount of [¹⁴C] or [³H]oxysterol as a fraction of total non-oxidized [³H]cholesterol in the sample (entire cholesterol pool).

FIG. 9 shows cholesterol biosynthesis (A) and esterification (B) in transfected and non-transfected cells. Cells were incubated in serum-free medium with [³H] acetate for 20 min (A) or with [¹⁴C]oleic acid (B) for 2 h at 37° C. in a CO₂ incubator. Cells were washed and lipids were extracted and analyzed by TLC as described.

FIG. 10 shows proliferation rates of transfected or non-transfected cells. Cells were seeded at the same density in 6-well plates and number of cells was counted every day. Closed circles—HepG2 cells, opened circles—HepG2/cav cells.

FIG. 11 shows the transfection of the RAW mouse macrophage cell line with CYP27. Red, in D, is the mitochondrial marker dye, green in C, is CYP27. A, B show poorly expressing cells, C, D show well expressing cells, E shows an overlap of C&D showing that CYP27 is in mitochondria.

FIG. 12 shows cholesterol efflux from transfected and mock-transfected cells to plasma and apoA-1.

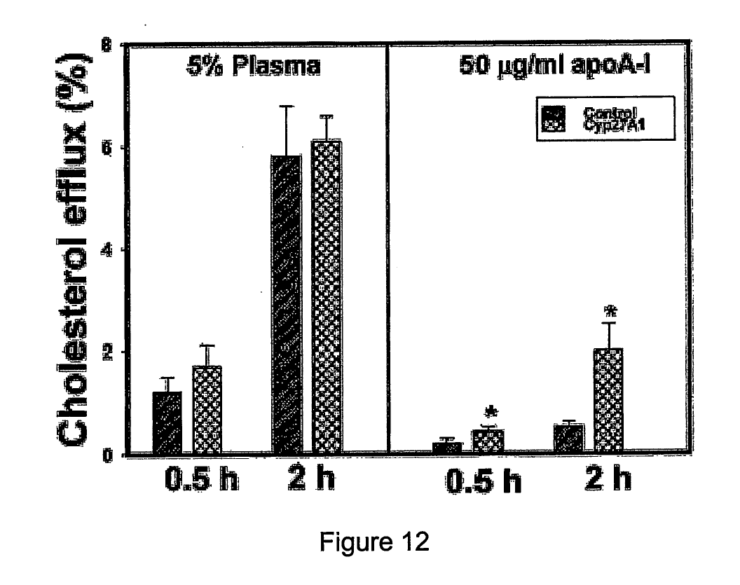

### DESCRIPTION OF THE INVENTION

In a first aspect of the present invention, there is provided a method of modulating cholesterol efflux in a cell, said method comprising
modulating expression and/or activity of sterol 27-hydroxylase (CYP27) and/or cavelolin-1 in the cell.

Sterol 27 hydroxylase (CYP27) is implicated in bile acid synthesis and is primarily found in hepatic cells. Hence its role is to convert cholesterol to bile acids in hepatic cells and thereby remove cholesterol from the cell in this manner. However, this enzyme is not commonly found in all cells and its role particularly in the macrophage and in epithelial cells is largely unknown. There is no requirement to produce bile in these cells and therefore the role of CYP27 is different. The enzyme has been found in macrophages and smooth muscle cells and was assumed to participate in the removal of cholesterol, generally by oxidation of cholesterol to more effluxable forms of cholesterol such as 27 hydroxy-cholesterol and 3-beta-hydroxy-5-cholestenoic acid. However, Applicants have found that cholesterol can be removed by cholesterol efflux which is regulated by CYP27. More surprisingly the cholesterol effluxed remains in the form of cholesterol and not as oxidized cholesterol as would be expected in the presence of CYP27.

In vessel walls, the reverse cholesterol transport pathway removes excess cholesterol, but the actual process of removal and the steps involved is complex. Applicants have found that cholesterol efflux from cells to acceptors such as apolipoprotein A-1 in plasma, mediated by sterol 27-hydroxylase, may contribute to the removal of cholesterol from the cells of the vessel wall and that the efflux of cholesterol is in an unoxidized form and is regulated by the enzyme CYP27.

Caveolin-1 is involved in pathways of intracellular cholesterol trafficking and cholesterol efflux and has been implicated in intracellular cholesterol trafficking. Being an essential component of caveolae, caveolin is also involved in numerous signaling pathways. Caveolae are specialized domains of the plasma membrane that are implicated in the sequestration of a variety of lipid and protein molecules. It has been suggested that these important cellular organelles have a pivotal role in such diverse biochemical processes as lipid metabolism, growth regulation, signal transduction, and apoptosis. Caveolin interacts with and regulates heterotrimeric G-proteins.

Currently, there are three members of the caveolin multigene family which are known to encode 21-24 kDa integral membrane proteins that comprise the major structural component of the caveolar membrane in vivo. Caveolin-2 protein is abundantly expressed in fibroblasts and differentiated adipocytes, smooth and skeletal muscle, and endothelial cells. The expression of caveolin-1 is similar to that of caveolin-2 while caveolin-3 expression appears to be limited to muscle tissue types. However, there are inconsistent reports on the connection of the level of caveolin expression and cholesterol effluxed. Moreover, attempts to stimulate cholesterol efflux by overexpression of caveolin were so far unsuccessful as it did not result in the formation or changes in the abundance of caveolae.

The cell in which cholesterol efflux is modulated may be any cell that can produce cholesterol and can export cholesterol by cholesterol efflux. Preferred cells include, but are not limited to, hepatic cells, macrophages, endothelial cells, smooth muscle cells and other cells of the vessel wall. Most preferably the cells are from a vessel wall that is associated with an atherosclerotic plaque such as a macrophage, or a hepatic cell that is associated with HDL formation. However, it is also contemplated in the present invention to use stem cells that may differentiate to somatic cells in which cholesterol is effluxed. Preferably the somatic cell is selected from hepatic cells, macrophages, endothelial cells and other vessel cells; preferably, the stem cell is a haematopoietic stem cell. More preferably, cholesterol efflux in non-hepatic cells is modulated by modulating expression and/or activity of CYP27. Cholesterol efflux in hepatic cells is preferably modulated by modulating expression and/or activity of caveolin-1. The mechanisms of cholesterol efflux and the benefits to the cells may be different in the various cell types. Cholesterol efflux generally has two functions. The first is to remove cholesterol from cells and protect against atherosclerosis by preventing accumulation of cholesterol. The second is to make high density lipoprotein (HDL) formed as a result of cholesterol efflux. HDL may then be atheroprotective by further reducing cholesterol in cells and by other mechanisms unrelated to cholesterol. The macrophage is a critical cell as this is where cholesterol accumulates with clinically significant consequences. The hepatocyte is a critical cell as this is where most of HDL is formed. Accordingly, it is preferred that the macrophage has modulated CYP27 and hepatocytes have modulated caveolin-1. These cells will benefit most from the modulation of CYP27 and/or caveolin-1. However, other cells may also benefit by the modulation of CYP27 and caveolin-1 alone or in combination.

Sterol-27-hydroxylase (CYP27) may be any form of the hydroxylase that oxidises cholesterol and converts cholesterol to 27 hydroxy cholesterol and 3-beta-hydroxy-5-cholestenoic acid. An “equivalent” as used herein is any compound that can behave and functions in a similar manner to CYP27 which may include various isoforms of the enzyme. The function of the enzyme may include its ability to oxidize cholesterol but also to contribute to any upstream or downstream reactions that can influence efflux of cholesterol

It is thought that the product of cholesterol oxidation by CYP27 regulates the efflux of un-oxidised cholesterol. It can either be a regulator of one of the genes involved in cholesterol efflux (or its regulation) or it can change the properties of the membranes (or a specific part of the membrane) to stimulate cholesterol efflux. The activity of CYP27 in the cells is rather low (even after transfection) and although almost all oxidised cholesterol is released, it consists of only about 2-3% of total (i.e. oxidised plus non-oxidised) cholesterol released. Thus the role of CYP27 in cholesterol oxidation does not change.

Throughout the description and claims of this specification, the word “comprise” and variations of the word, such as “comprising” and “comprises”, is not intended to exclude other additives, components, integers or steps.

Caveolae are considered to be a principle source of cholesterol for efflux and caveolin is involved in transporting cholesterol from intracellular compartments to a plasma pool membrane where it becomes accessible for efflux. Caveolin is expressed in many, but not all cell types and may or may not result in formation of caveolae. It is a preferred aspect of this invention that overexpressing caveolin-1 in hepatic cells does result in increased cholesterol efflux and preferably formation of caveolae which stimulates cholesterol efflux. Cholesterol efflux from hepatic cells is the major source of HDL cholesterol and stimulation of cholesterol efflux from these cells would increase plasma levels of HDL, which is anti-atherogenic.

Cholesterol efflux is the first step of RCT and the liver is usually considered a destination rather than an origin of RCT. However, the rate of cholesterol efflux from hepatic cells such as HepG2 cells is very similar to that from human skin fibroblasts implying that properties of cholesterol efflux pathway are similar between these cell types. However, Hep G2 cells do not naturally express caveolin-1. Further, it was recently suggested that liver may be the major source of HDL cholesterol in plasma. Thus, it has now been found that liver may play a dual role in regulation of RCT by being both origin and destination of the pathway.

The present invention also includes the use of both mechanisms to improve the modulation of the cholesterol efflux. Accordingly, the cholesterol efflux of the cell may be modulated by modulating the expression and/or activity of CYP27 and caveolin-1 or equivalents alone or in combination. Cholesterol efflux may be enhanced by modulating CYP27 in one cell and caveolin-1 in another cell and by expressing both, they may have a synergistic effect wherein one provides cholesterol efflux and the other HDL which in turn enhances the cholesterol efflux and clearing from the cells.

The term “modulating” cholesterol efflux as used herein means to alter the rate of which cholesterol is removed from the cell. The degree of alteration is determined by comparing against natural rates of cholesterol efflux from the cell. “Modulating” includes adjusting cholesterol efflux to increase or decrease efflux. Preferably, the efflux is increased. Hence by targeting CYP27 and caveolin-1, cholesterol efflux can be manipulated.

The discovery in the present application that CYP27 and caveolin-1 are central to cholesterol efflux wherein the efflux is substantially in the form of unmodified cholesterol has provided a means by which cholesterol efflux can be changed, altered or adjusted as required. By “substantially” we mean predominantly in the form of non-oxidized cholesterol rather than the oxidized forms including 27 hydroxy cholesterol and 3-beta-hydroxy-5-cholestenoic acid. When cholesterol is effluxed, some is effluxed in this oxidized form. However, when regulated or modulated by CYP27, cholesterol in non-oxidized form is predominantly effluxed.

The amount of cholesterol effluxed from the cell will be dependent upon the conditions imposed on the cell. Preferably, 5-15% of cholesterol in the cell is released from the cell. However, these amounts may vary under different conditions. The form of cholesterol released may be in an oxidised or non-oxidised form. Preferably, of the 5-15%, approximately 0.1-0.3% may be released in an oxidised form and the remaining portion released as a non-oxidised form. However, since there is very little oxidised cholesterol in the cell (2-3%), the relatively small amount of oxidised cholesterol released may comprise about 80% of the amount of oxidised cholesterol formed by CYP27.

The term “modulating expression and/or activity of CYP27” or “modulating expression and/or activity of caveolin-1” as used herein includes modifying or altering the expression and/or activity of CYP27 and/or caveolin-1 compared to unmodified levels of CYP27 and/or caveolin-1. Expression and/or activity may be increased or decreased compared to unmodified levels to increase or decrease cholesterol efflux. Preferably, expression and/or activity of CYP27 and/or caveolin-1 is increased to increase cholesterol efflux.

Modulation of CYP27 and/or caveolin-1 expression and/or activity may be achieved by direct or indirect methods. Modulation of expression and/or activity of CYP27 and/or caveolin-1 may be achieved using direct methods known to those of skill in the art and include, but are not limited to, introduction of genes encoding CYP27 and/or caveolin-1, knockout technology, antisense technology, triple helix technology, targeted mutation, gene therapy and regulation by agents acting on transcription. Indirect methods for modulating expression and/or activity of CYP27 and/or caveolin-1 may include targeting upstream or downstream regulators.

“Activity” as used herein relates to a function of a CYP27 and/or caveolin-1 in a cell, and includes the ability of CYP27 and/or caveolin-1 and/or products of the reaction catalysed by CYP27 and/or caveolin-1 to bind to chaperone, or upstream or downstream effector molecules thereby activating or repressing upstream or downstream pathways which affect cholesterol efflux. The term “activity” also includes the ability of CYP27 to oxidize cholesterol and/or caveolin-1 to efflux cholesterol.

In a further preferred aspect of the present invention, there is provided a method of modulating cholesterol efflux in a cell, said method comprising:
transfecting the cell with a gene encoding CYP27 and/or caveolin-1; and
modulating expression and/or activity of the transfected CYP27 and/or caveolin-1 in the cell.

This method of modulating cholesterol efflux by directly affecting expression of CYP27 and/or caveolin-1 is preferentially used to modulate CYP27 and/or caveolin-1 expression from the natural levels. Preferably, the introduction of a construct with the gene for CYP27 and/or caveolin-1 will increase the level of CYP27 and/or caveolin-1 upon expression and thereby affect the cholesterol efflux.

As discussed above, any cell that can efflux cholesterol can accept a gene encoding CYP27 and/or caveolin-1. However, ideally, the cell is a cell that requires efficient efflux of cholesterol or is involved in HDL formation. All cells efflux cholesterol, but cells of the vessel wall are most preferred for CYP27 and are those where this matters most because impaired cholesterol efflux in these cells leads to atherosclerosis. Hepatic cells are most preferred for caveolin-1 as they are involved in HDL formation. Stem cells are also considered within the scope of the present invention. These will be particularly useful for gene therapy and upon differentiation can be modulated in the appropriate regions of the body such as in vessel walls, cardiac tissue or liver to effectively cause cholesterol efflux upon further expression of CYP27 and/or caveolin-1.

The gene for CYP27 may be obtained from the Baker Medical Research Institute and inserted into a mammalian expression vector such as pcDNA3 to form a construct or vector that may be transfected into the cell to express CYP27.

The gene for caveolin-1 may be obtained from any commercial source such as from Clontech or as an expression vector that can be transfected into the cells. However, any method of introducing the gene sequence into the cell is within the scope of the present invention.

Preferably, a gene sequence for CYP27 and/or caveolin-1 is operably linked to a regulatory sequence which is capable of providing for the expression of the coding sequence by a cell. The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

The term “regulatory sequences” includes promoters and enhancers and other expression regulation signals. These may be selected to be compatible with the cell for which the expression vector is designed. Mammalian promoters, such as β-actin promoters and the myosin light chain promoter may be used. However, other promoters may be adopted to achieve the same effect. These alternate promoters are generally familiar to the skilled addressee. Mammalian promoters also include the metallothionein promoter which can upregulate expression in response to heavy metals such as cadmium and is thus an inducible promoter. Tissue-specific promoters may be used. Viral promoters may also be used, for example the Moloney murine leukaemia virus long terminal repeat (MMLV LTR), the promoter rous sarcoma virus (RSV) LTR promoter, the SV40 promoter, the human cytomegalovirus (CMV) IE promoter, herpes simplex virus promoters or adenovirus promoters. All these promoters are readily available in the art.

Such vectors may be transfected into a suitable cell in which cholesterol efflux or HDL formation is desired to provide for expression of a polypeptide encoding CYP27 and/or caveolin-1.

The vectors may be, for example, plasmid, virus or phage vectors provided with an origin of replication, optionally a promoter for the expression of the CYP27 and/or caveolin-1 and optionally a regulator of the promoter. The vectors may contain one or more selectable marker genes for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo for example in a method of gene therapy or a DNA vaccine.

Most preferably the pcDNA3 vector is used for the expression of CYP27. Preferably the vector contains the G418 (geneticin) resistance gene.

For the expression of caveolin-1, it is preferred to use the plasmid pIRES2-EGFP/caveolin-1 (Clontech) plasmid.

The cells in which the vector is transfected is expected to provide for such post-translational modifications (eg methylation, myristolation, glycosylation, truncation, lapidation and tyrosine, serine or threonine phosphorylation) as may be needed to confer optimal biological activity on recombinant expression products.

Preferably, the cells may be exposed to a demethylation agent to prevent supression of the expression of CYP27. More preferably, 5-azacytidine is used as a demethylating agent. However, any demethylating agent can be used.

The vector may be transfected into the cell by any means available to the skilled addressee. Preferably, the vector is introduced by calcium phosphate precipitation, electroporation, biolistics, lipofection, DEAE Dextran or adenoviral or retroviral infection. However, this invention is not restricted to these methods.

The expression of CYP27 and/or caveolin-1 may be increased to a level above the normal CYP27 and/or caveolin-1 expression to favour and enhance cholesterol efflux or HDL formation. The degree of enhancement may be measured by actual cholesterol effluxed such as, but not limited to measuring the release of labelled cholesterol when cellular cholesterol or a specific pool of it is labelled prior to the efflux experiment or measuring of cholesterol mass in cells and/or medium by commercially available fluorometric assays.

In a preferred aspect there is provided a method of increasing cholesterol efflux in a cell, said method comprising
increasing expression and/or activity of CYP27 and/or caveolin-1 in the cell.

By increasing the expression and/or activity of CYP27 and/or caveolin-1 in the cell, it has now been found that cholesterol efflux can be increased. In the case of CYP27, the increase in cholesterol efflux is not due to increased efflux of oxidized cholesterol, but of mostly non-oxidized cholesterol. Quite unexpectedly, applicants have found that increasing CYP27 expression and/or activity does not lead to marked increases in oxidation products. The small amount of oxidized cholesterol is very low and not toxic at this concentration. High concentrations would generally be expected to be toxic. However, there is a marked increase in the efflux of non-oxidized cholesterol from the cell.

In a further preferred aspect of the present invention, there is provided a method of increasing cholesterol efflux from a cell, said method comprising
transfecting the cell with a gene encoding CYP27 and/or caveolin-1; and
increasing expression of the transfected CYP27 and/or caveolin-1.

Modulation of CYP27 and/or caveolin-1 to increase expression and/or activity may be achieved by inducing expression of CYP27 and/or caveolin-1 by transfection of a construct containing the CYP27 and/or caveolin-1 gene or by overexpressing the gene in the cell. By introduction of an exogenous CYP27 and/or caveolin-1 or a construct to express exogenous CYP27 and/or caveolin-1, the ability of CYP27 and/or caveolin-1 to increase cholesterol efflux may be modulated. Cholesterol efflux may also be increased further in this setting by cotransfection with a plasmid expressing apolipoprotein A1, apolipoprotein E or by introduction of these apolipoproteins.

The cells are preferably transfected with CYP27 and/or caveolin-1 by any means that introduces a CYP27 and/or caveolin-1 gene to the cell. Preferably, the gene encoding CYP27 and/or caveolin-1 is transfected into the cell via an expression vector by methods available to the skilled addressee or as described above.

Preferably a construct of CYP27 and/or caveolin-1 is introduced or transfected into the cell to increase the expression of CYP27 and/or caveolin-1 thereby increasing cholesterol efflux. Increasing the expression may be achieved by any means known to the skilled addressee including the induction of promoters in the construct. Vectors may be used with regulatory regions that respond to tetracycline, mifepristone or ecdysone.

The construct and the cells in which the construct is transfected is expected to undergo such post-translational modifications (eg methylation, myristolation, glycosylation, truncation, lapidation and tyrosine, serine or threonine phosphorylation) as may be needed to confer optimal biological activity on recombinant expression products.

The cells may be further treated with a demethylating agent such as, but not limited to 5-azacytidine to enhance the expression of CYP27.

However, the expression and/or activity may also be increased by indirect methods of targeting indirect regulators to upregulate the gene. These regulators may act on the promoters that cause expression of the gene or they may act on upstream or downstream molecules that affect the enzyme. For instance adrenodoxin and adrenodoxin reductase may be targeted because they are important for the flow of electrons during the CYP27 enzymatic reaction. In transfection studies, fusion of all three proteins into a single chimera, or transfected in tandem, may generate more enzymatic activity than addition of the CYP27 gene alone. The chimera would be the molecule of choice for gene therapy work. It may be delivered in a vector with a promoter containing regulatory regions which preferably respond to metals, tetracycline, mifepristone or ecdysone. The chimera may also be delivered in tandem with a vector expressing another protein which has been shown to increase cholesterol efflux, such as, but not limited to apolipoprotein A1. The endogenous gene may be regulated by dexamethasone which increases CYP27 expression. Promoter studies show that CYP27 is upregulated by dexamethasone and downregulated by cyclosporin A and cholic acid. Accordingly, dexamethasone and cyclosporin A and cholic acid may be successfully used to regulate CYP27 expression.

Caveolin-1 expression may also be regulated by co-expression with caveolin-2, by SREBP (Sterol Regulatory Element Binding Protein) or phosphorylation. By targeting any one of these regulators, expression of caveolin-1 can be modulated.

Regulation of the gene expression may generally be achieved by the use of molecules reacting with the promoter of the gene or with a promoter of a nuclear factor regulating the gene, or by RNA processing including splicing and degradation. The activity of enzyme itself may also be targeted by phosphorylation, or allosteric regulation or regulation of the enzyme degradation such as by the use of protease inhibitors.

Increased expression and/or activity of CYP27 and/or caveolin-1 may be achieved by any means that can increase endogeneous CYP27 and/or caveolin-1 expression and/or activity thereby resulting in increased cholesterol efflux.

Preferably, the increased cholesterol efflux is via a mechanism which involves transfer of cholesterol from plasma membrane caveolae. Preferably, this method of cholesterol efflux induces a “fast” efflux of cholesterol by releasing cholesterol from more accessible sites of the plasma membrane. Accordingly, in another aspect of the present invention there is provided a method of increasing cholesterol efflux from caveolae, said method comprising:
increasing expression and/or activity of CYP27 and/or caveolin-1 in the cell.

More preferably, the increased expression and/or activity is induced by transfection of the cell with CYP27 and/or caveolin-1. The expression of the CYP27 and/or caveolin-1 is therefore increased to increase cholesterol efflux. Cholesterol efflux may also be increased further in this setting by cotransfection with a plasmid expressing apolipoprotein A1 or by introduction of apolipoprotein, as previously described.

Alternatively, modulation of CYP27 and/or caveolin-1 to decrease expression and/or activity in the cell may be achieved using antagonists, inhibitors, mimetics or derivatives of CYP27 and/or caveolin-1. The terms “antagonist” or “inhibitor”, as used herein, refer to a molecule which, when bound to CYP27 and/or caveolin-1, blocks or modulates the activity of CYP27 and/or caveolin-1. Antagonists and inhibitors may include proteins, nucleic acids, carbohydrates, antibodies or any other molecules including ligands which bind to CYP27 and/or caveolin-1. Other modulators of the activity and/or expression of CYP27 and/or caveolin-1 include a range of rationally-designed, synthetic inhibitors.

Cholesterol efflux has been implicated as a potent regulator of cell growth: the less efflux the more growth and vice versa. Accordingly, decreases in cholesterol efflux may be useful in applications including re-stenosis after angioplasty, cancer treatment for inhibition of growth by stimulating efflux or regeneration by stimulation of growth by inhibition of the efflux.

In another aspect of the present invention there is provided a cell with modulated cholesterol efflux, said cell having modulated CYP27 and/or caveolin-1 expression and/or activity.

Preferably the cell is transfected with a gene encoding CYP27 and/or caveolin-1. The cell is therefore capable of modulation, preferably having increased cholesterol efflux upon increased CYP27 and/or caveolin-1 expression. Cholesterol efflux may also be increased further by cotransfection with a plasmid expressing apolipoprotein A1 or apolipoprotein E or by introduction of these apolipoproteins.

The cell may be any cell that can efflux cholesterol. Preferably the cell is selected from the group including, but not limited to, hepatic cells, macrophages, vessel cells, endothelial cells, smooth muscle cells or stem cells. Preferably the stem cells are capable of differentiating to any of the cells selected from the group including hepatic cells, macrophages, endothelial cells and other vessel cells. Preferably, the stem cells are hematopoietic stem cells. Hepatic cells are cells producing most of plasma HDL and responding to overexpression of caveolin and therefore it is most preferred to introduce caveolin-1 into hepatic cells to increase cholesterol efflux in these cells.

In yet another aspect of the present invention, there is provided a method of treating a cholesterol related condition in a patient by modulating cholesterol efflux from a cell of the patient, said method comprising:
modulating expression and/or activity of CYP27 and/or caveolin-1 in the cell.

The term “treatment” is used herein in its broadest sense to include preventative treatments or treatment that are intended to prevent further progression of the condition. In this respect, the treatment may include modulating CYP27 and/or caveolin-1 expression and/or activity at a level to prevent further progression of the cholesterol related condition by affecting the cholesterol efflux from the cell.

A “cholesterol related condition” as used herein may include any vascular conditions, including myocardial infarction, atherosclerosis, stroke, hypoalphalipoproteinaemia or peripheral vascular disease. Generally, these conditions result from an accumulation of cholesterol in plaques thereby causing narrowing of the vessel lumen in vessels, such as blood vessels, resulting in the condition which often manifests in tissue damage or death. Preferably the cholesterol related condition is atherosclerosis.

Preferably, the modulation of the expression and/or activity of CYP27 and/or caveolin-1 is increased to increase cholesterol efflux.

The expression and/or activity of CYP27 and/or caveolin-1 may be modulated separately or in combination in one cell type or several cell types. Modulation of expression and/or activity of CYP27 is preferably to modulate cholesterol efflux of non-oxidized cholesterol. A useful cell type in which cholesterol efflux is modulated is a macrophage. When modulated in combination with caveolin-1, the cholesterol efflux may be enhanced. Since caveolin-1 is actively involved in HDL formation, the combination of cholesterol efflux and HDL formation can reduce the build-up of cholesterol to treat a cholesterol-related condition. Preferably, the caveolin-1 is modulated in a hepatic cell.

In an even further preferred aspect of the present invention, there is provided a method of treating a cholesterol related condition in a patient by modulating cholesterol efflux in a cell in the patient, said method comprising:
introducing a gene construct to modulate expression and/or activity of CYP27 and/or caveolin-1 in the cell in the patient.

The present invention also encompasses gene therapy whereby a gene encoding CYP27 and/or a CYP27 regulator and/or caveolin-1 and/or a caveolin-1 regulator, is regulated in a patient. Various methods of transferring or delivering DNA to cells for expression of the gene product protein, otherwise referred to as gene therapy, are disclosed in Gene Transfer into Mammalian Somatic Cells in vivo, N. Yang, Crit. Rev. Biotechn. 12(4): 335-356 (1992), which is hereby incorporated by reference.

Preferably, cholesterol efflux may also be increased further by cotransfection with a plasmid expressing apolipoprotein A1 or apolipoprotein E or by introduction of these apolipoproteins.

Preferably, CYP27 and caveolin-1 are modulated alone or in combination. More preferably, they are modulated in combination in separate cells. Most preferably, they are modulated in macrophages and hepatocytes.

Strategies for treating these medical problems with gene therapy include therapeutic strategies such as identifying a defective gene or protein and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene such as that for CYP27 and/or a CYP27 regulator and/or caveolin-1 and/or a caveolin-1 regulator may be placed in a patient and thus prevent occurrence of atherosclerosis or a related condition; or a gene that makes a cell more susceptible to modulation of cholesterol efflux.

Many protocols for transfer of CYP27 DNA, or CYP27 regulatory sequences and/or caveolin-1 DNA or caveolin-1 regulatory sequences are envisioned in this invention. Transfection of promoter sequences, or other sequences which would modulate the expression and/or activity of CYP27 and/or caveolin-1 are also envisioned as methods of gene therapy. An example of this technology is found in Transkaryotic Therapies, Inc., of Cambridge, Mass., using homologous recombination to insert a “genetic switch” that turns on an erythropoietin gene in cells. See Genetic Engineering News, Apr. 15, 1994. Such “genetic switches” could be used to activate CYP27 (or CYP27 regulators) and/or caveolin-1 (or caveolin-1 regulators) in a cell.

Gene transfer methods for gene therapy fall into three broad categories: physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer.

Chemical methods of gene therapy may involve a lipid based compound, not necessarily a liposome, to ferry the DNA across the cell membrane. Lipofectins or cytofectins, lipid-based positive ions that bind to negatively charged DNA, may be used to cross the cell membrane and provide the DNA into the interior of the cell. Another chemical method may include receptor-based endocytosis, which involves binding a specific ligand to a cell surface receptor and enveloping and transporting it across the cell membrane.

Many gene therapy methodologies employ viral vectors such as retrovirus vectors to insert genes into cells. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors may be selected from the group including, but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors and are preferred. Adenoviral vectors may be delivered bound to an antibody that is in turn bound to collagen coated stents.

Mechanical methods of DNA delivery may be employed and include, but are not limited to, fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” inorganic chemical approaches such as calcium phosphate transfection and plasmid DNA incorporated into polymer coated stents. Ligand-mediated gene therapy, may also be employed involving complexing the DNA with specific ligands to form ligand-DNA conjugates, to direct the DNA to a specific cell or tissue.

The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Gene regulation of CYP27 and/or CYP27 regulators and/or caveolin-1 and/or a caveolin-1 regulator may be accomplished by administering compounds that bind to CYP27 and/or caveolin-1 genes, or control regions associated with the CYP27 and/or caveolin-1 genes, or corresponding RNA transcript to modify the rate of transcription or translation. Additionally, cells transfected with a DNA sequence encoding CYP27 and/or CYP27 regulators and/or caveolin-1 and/or a caveolin-1 regulator may be administered to a patient to provide an in vivo source of CYP27 and/or a CYP27 regulator and/or caveolin-1 and/or a caveolin-1 regulator. For example, cells may be transfected with a vector containing a nucleic acid sequence encoding CYP27 and/or a CYP27 regulator and/or caveolin-1 and/or a caveolin-1 regulator. The term “vector” as used herein means a carrier that can contain or associate with specific nucleic acid sequences, which functions to transport the specific nucleic acid sequences into a cell. Examples of vectors include plasmids and infective microorganisms such as viruses, or non-viral vectors such as ligand-DNA conjugates, liposomes, lipid-DNA complexes. It may be desirable that a recombinant DNA molecule comprising a CYP27 and/or CYP27 regulator and/or caveolin-1 and/or a caveolin-1 regulator DNA sequence is operatively linked to an expression control sequence to form an expression vector capable of expressing CYP27 and/or a CYP27 regulator and/or caveolin-1 and/or a caveolin-1 regulator. The transfected cells may be cells derived from the patient's normal tissue, the patient's diseased tissue, or may be non-patient cells. For example, blood, hepatic or vessel cells removed from a patient can be transfected with a vector capable of expressing CYP27 and/or a CYP27 regulator and/or caveolin-1 and/or a caveolin-1 regulator of the present invention, and be re-introduced into the patient. The transfected cells demonstrate modulated CYP27 and/or caveolin-1 expression and/or activity in the patient that modulate cholesterol efflux. Patients may be human or non-human animals. Cells may also be transfected by non-vector, or physical or chemical methods known in the art such as electroporation, incorporation, or via a “gene gun.” Additionally, CYP27 and/or CYP27 regulator and/or caveolin-1 and/or a caveolin-1 regulator DNA may be directly injected, without the aid of a carrier, into a patient. In particular, CYP27 and/or CYP27 regulator and/or caveolin-1 and/or a caveolin-1 regulator DNA may be injected into blood.

The gene therapy protocol for transfecting CYP27 and/or a CYP27 regulator and/or caveolin-1 and/or a caveolin-1 regulator into a patient may either be through integration of CYP27 and/or CYP27 regulator and/or caveolin-1 and/or a caveolin-1 regulator DNA into the genome of the cells, into minichromosomes or as a separate replicating or non-replicating DNA construct in the cytoplasm or nucleoplasm of the cell. Modulation of CYP27 and/or caveolin-1 expression and/or activity may continue for a long-period of time or may be reinjected periodically to maintain a desired level of CYP27 and/or caveolin-1 expression and/or activity in the cell, the tissue or organ.

In a further preferred aspect of the present invention, there is provided a method of treating a cholesterol related condition in a patient by modulating cholesterol efflux in the patient, said method comprising:
introducing modulated cells to the patient, wherein said cells have modulated expression and/or activity of CYP27 and/or caveolin-1.

The modulated cells are intended to replace existing cells such that the existing cholesterol efflux of the cells is modulated or the modulated cells may be used to infiltrate existing regions of disease such as in an atheroma to halt progression of the atheroma. Preferably, the cholesterol efflux is increased by increasing expression and/or activity of CYP27 and/or caveolin-1. More preferably, the expression and/or activity of CYP27 and/or caveolin-1 is increased by transfection of a gene encoding CYP27 and/or caveolin-1 to the cells. The gene may then be over expressed or turned on to increase expression of the CYP 27 and/or caveolin-1.

The replaced cells may be any cell that can efflux cholesterol and may be tissue specific for the condition to be treated. Preferably the cell is selected from the group including, but not limited to, hepatic cells, macrophages, vessel cells, endothelial cells or stem cells. Preferably the stem cells are capable of differentiating to any of the cells selected from the group including hepatic cells, macrophages, vessel cells, or endothelial cells. Preferably, the stem cells are hematopoietic stem cells. It is preferred that when using stem cells the cells may be transfected with a gene that encodes CYP27 and/or caveolin-1, such that upon implantation to a tissue requiring treatment, the cells may differentiate to the cells of the region. Regulators modulating sequences of a vector that has been introduced to the cell can switch expression of CYP27 and/or caveolin-1 on or off accordingly to the level of cholesterol efflux. These cells will eventually replace diseased cells that have accumulated cholesterol.

Preferably, the modulated cells are single cell types wherein the CYP27 and the caveolin-1 are both modulated. More preferably, the modulated cells are different cell types that are receptive to modulation of CYP27 or caveolin-1 so as to enhance cholesterol efflux from the cells. Preferably, the macrophage and/or smooth muscle cells express modulated CYP27 expression and/or activity. Also, it is preferred that hepatic cells express modulated caveolin-1 expression and/or activity.

Preferably the cell that is targeted is directly related to the condition, such as but not limited to vessel cells and macrophages found in the vessel wall and are directly related to conditions such as atherosclerosis.

In yet another aspect of the present invention there is provided a method of identifying a compound which modulates cholesterol efflux in a cell, said method comprising:
contacting the compound to the cell;
detecting a change in CYP27 and/or caveolin-1 expression and/or activity in the cell relative to a cell which has not been contacted with the compound.

The present applicants have identified a target which provides an indication of cholesterol efflux in the cells. Cholesterol efflux is regulated by CYP27 and/or caveolin-1. Hence compounds which can modulate a change in CYP27 and/or caveolin-1 can influence cholesterol efflux.

Preferably the cell is a cell in which cholesterol efflux can be readily measured. Preferred cells include hepatic cells, endothelial cells, smooth muscle cells and other cells of the vessel wall. Cells that have been transfected with a gene encoding CYP27 and/or caveolin-1 may also be used. By monitoring the expression of the introduced CYP27 and/or caveolin-1, the effect of the compound on the expression can be used to determine compound that may affect cholesterol efflux. Preferably, the cell is a macrophage or a hepatocyte. More preferably the cell is a hepatocyte transfected with caveolin-1. More preferably, the cell is a transfected HepG2 cell.

Measuring cholesterol efflux may be conducted by any means which can measure the movement of a compound such as cholesterol in a cell. Methods such as labelling the cholesterol with radioactive labels maybe utilised. However, these methods are familiar to the skilled addressee.

The contacting of the cell to the unknown compound may be by any means dependent upon the characteristics of the compound providing the cell can interact and be influenced by the compound to be tested. For instance, if the compound is soluble, this would involve direct contacts with the cell. However, if insoluble, suspensions of the compounds may be used.

These skills will be familiar to the person testing the compound.

The identification of the compound which can modulate cholesterol efflux will depend on its effect of changing expression and/or activity of CYP27 and/or caveolin-1 in the cell.

Preferably, the change increases expression and/or activity of CYP27 and/or caveolin-1 in the cell to identify a compound which increases cholesterol efflux. Alternatively, it is preferred that the change decreases expression and/or activity of CYP27 and/or caveolin-1 in the cell to decrease cholesterol efflux from the cell. Compounds may therefore be selected based on the effect on CYP27.

Methods of detecting the change in expression and/or activity of CYP27 and/or caveolin-1 may include measurement of the genetic expression of CYP27 and/or caveolin-1 including DNA, RNA, mRNA expression in the cell. Measurement of changes in these parameters in the cell are familiar to the skilled person.

Measurement of CYP27 and/or caveolin-1 by the use of antibodies may also be used to identify a change in CYP27 and/or caveolin-1 in the presence of absence of the unknown compound that is being tested.

Activity of CYP27 can be measured by the increase or decrease of oxidising cholesterol to convert cholesterol to 27 hydroxy cholesterol and 3-beta-hydroxy-5-cholestenoic acid.

Once a compound is identified to change CYP27 and/or caveolin-1 expression and/or activity then it may be utilised in the manufacture of a medicament to treat cholesterol related conditions. Accordingly, the invention also provides a method of obtaining a composition when used for treating a cholesterol related condition, said method comprising:
providing a compound in an amount effective to treat the condition wherein said compound is identified by the methods described above; and
admixing the compound with a pharmaceutically acceptable carrier.

A pharmaceutically acceptable carrier may be any carrier known to the skilled addressee which is not toxic to the patient and which can be admixed to form a pharmaceutical.

Examples of the procedures used in the present invention will now be more fully described. It should be understood, however, that the following description is illustrative only and should not be taken in any way as a restriction on the generality of the invention described above.

### EXAMPLES

### Example 1

### Expression of Sterol 27-hydroxylase (CYP27A1) Enhances Cholesterol Efflux

1. Methods

a) Cells.

CHOP-C4 cells were grown in RPMI 1640 medium containing 10% FBS, 2 mM L-glutamin, penicillin/streptomycin (50 units/ml) and 0.2 mg/ml G418. The day before transfection, cells were plated in 12 well plates at a density of 0.6×105 cells per well. Transfection was performed with DEAE-Dextran as described in Albiston, A. L., Obeyesekere, V. R., Smith, R. E., and Krozowski, Z. S. (1994) Mol. Cell. Endocrinol. 105, R11-17 using 200 ng of plasmid DNA (CYP27A1 in pcDNA1 or pcDNA1 alone) per well. CYP27A1 was isolated from a human kidney cDNA library during studies of progesterone metabolism using a screening protocol previously described for the isolation of 11 βHSD2 (Albiston, A. L et al (1994)).

b) Cholesterol Acceptors.

Blood from healthy normolipidemic volunteers was collected in saline containing streptokinase (Sigma, final concentration 150 units/ml) and plasma was isolated by repeated centrifugation for 15 min at 3000 rpm at 40° C. Apolipoprotein A-I was isolated as described previously in Morrison, J. R., Fidge, N. H., and Grego, B. (1990) Anal. Biochem. 186, 145-152.

c) Cholesterol Efflux.

Two methods were used to label cellular cholesterol. Metabolic labeling was conducted by incubating cells in serum-free medium with [1-¹⁴C] acetate (Amersham-Pharmacia-Biotech (APB), specific radioactivity 2.07 GBq/mmol, final radioactivity 0.4 MBq/ml) for 24 h at 37° C. in a CO₂ incubator. Alternatively, cells were incubated in serum containing medium with [1α,2α(n)-3H]cholesterol (APB, specific radioactivity 1.81 TBq/mmol, final radioactivity 0.2 MBq/ml) for 48 h in a CO₂ incubator. After labeling, cells were washed six times with PBS and further incubated for 2 h or the indicated periods of time at 37° C. with serum-free medium containing either lipid-free apoA-I (final concentration 30 μg/ml), or the indicated concentrations of human serum. The medium was then collected, centrifuged for 15 min at 40° C. at 30,000 g to remove cellular debris and the supernatant counted or used for further analysis. Cells were harvested using a cell scraper, dispensed in 0.5 ml distilled water and aliquots were counted or used for further analysis. Cholesterol efflux is expressed as a percentage of labeled cholesterol transferred from cells to the medium.

d) Lipid Analysis.

Lipids were extracted with 3 volumes ethylacetate and separated using TLC (chloroform/ethylacetate 4:1 v/v) (Penning, T. M., Burczynski, M. E., Jez, J. M., Hung, C. F., Lin, H. K., Ma, H., Moore, M., Palackal, N., and Ratnam, K. (2000) Biochem. J. 351, 67-77). TLC plates containing labeled lipids were exposed to a phosphoimager plate and analyzed on the Bioimager BAS-1000 (Fuji) and the radioactivity in each spot quantified. Spots of cholesterol and 27-hydroxycholesterol identified by standards (Research Plus, USA) were scraped and counted.

e) Northern Blotting.

RNA was separated on a 1.2% agarose gel, transferred to a nylon membrane and probed with ³²P-labeled mouse ABCA1 cDNA (Chambenoit, O., Hamon, Y., Marguet, D., Rigneault, H., Rosseneu, M., and Chimini, G. (2001) J. Biol. Chem. 276, 9955-9960) (gift of Dr. G. Chimini) and ³²P-labeled GAPDH cDNA as an internal standard. The membrane was exposed to a phosphoimager plate and analyzed on the Bioimager BAS-1000 (Fuji).

f) Statistical Analysis.

The Student's t-test was used to determine statistically significant differences between groups.

2. Results

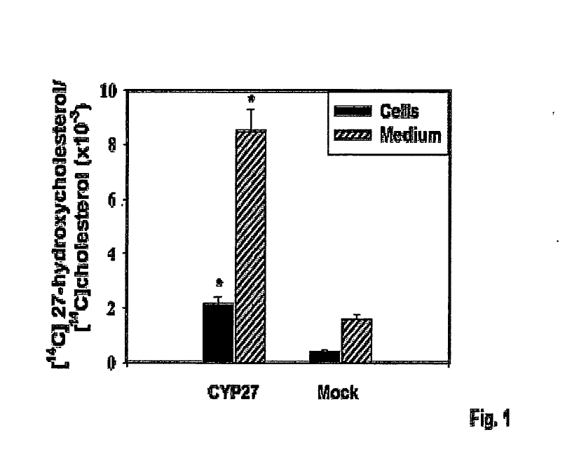

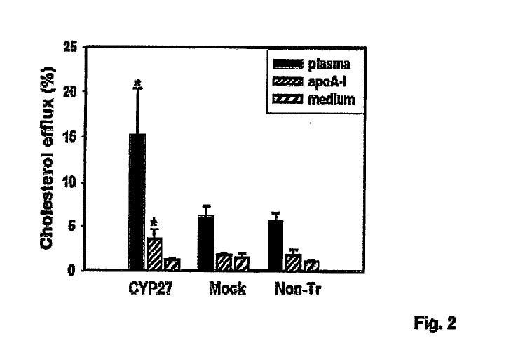

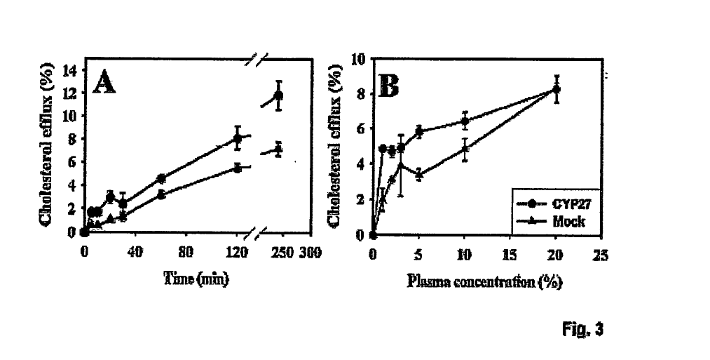

Transfection of CHOP cells with 27-hydroxylase (CYP271) resulted in production of 27-hydroxycholesterol when cells were metabolically labeled with [¹⁴C] acetate. The amount of newly synthesized 27-hydroxycholesterol found in transfected cells was 5-fold of that in non-transfected cells, however it was only 1% of the amount of newly synthesized cholesterol found in transfected cells (FIG. 1). When transfected cells were exposed to plasma for two hours, 80% of the newly synthesized 27-hydroxycholesterol, (FIG. 1) and 45% of newly synthesized cholesterol (not shown) was released to plasma. The contribution of 27-hydroxycholesterol to the overall amount of sterol released to plasma was therefore 1.5%. Thus although 27-hydroxycholesterol was released from cells more readily than cholesterol, its contribution to overall sterol efflux was minimal. When the cellular cholesterol pool was labeled with [³H] cholesterol, no formation of 27-hydroxycholesterol in either transfected or non-transfected cells was detected (not shown). To study the effect of transfection with CYP27 on cholesterol efflux, cellular cholesterol was labeled with [³H] cholesterol and cells incubated in the presence or absence of whole human plasma, or lipid-free human apoA-I. Transfection of cells with CYP27 resulted in a 3-fold increase in cholesterol efflux to whole plasma, and a doubling of the efflux to apoA-I compared to both non-transfected and mock transfected cells (FIG. 2). Cholesterol efflux to the medium without acceptors was not affected by transfection. When lipids released to the medium were analyzed, no 27-hydroxycholesterol was found and all released radioactivity was accounted for in the cholesterol fraction (not shown). The time-course of cholesterol efflux to human plasma is shown in FIG. 3A. The kinetics consists of “fast” and “slow” phases as reported by Gaus, K., Gooding, J. J., Dean, R. T., Kritharides, L., and Jessup, W. (2001) Biochemistry 40, 9363-9373). Transfection of cells with CYP27 resulted in a significant stimulation of the “fast” phase of the efflux with modest stimulation of the “slow” phase. Dose-dependence of the efflux is shown in FIG. 3B. Relatively more cholesterol was released from transfected cells at low plasma concentration, and the difference between transfected and non-transfected cells gradually disappeared when concentration of plasma increased. 27-hydroxycholesterol is a ligand of LXR receptor regulating a number of genes involved in lipid metabolism including ABCA1 (Fu, X., Menke, J. G., Chen, Y., Zhou, G., MacNaul, K. L., Wright, S. D., Sparrow, C. P., and Lund, E. G. (2001) J. Biol. Chem. 276, 38378-38387). To investigate if formation of 27-hydroxycholesterol lead to changes in the expression of ABCA1, the expression of ABCA1 in transfected and non-transfected cells was evaluated. ABCA1 was identified on the Northern blot using mouse ABCA1 cDNA probe as a single band (not shown). There was little difference in the abundance of ABCA1 mRNA between transfected and mock-transfected cells. The ratio of ABCA1 to GAPH mRNA was 0.84 and 1.36 for transfected and mock-transfected cells, respectively. To evaluate the amount of ABCA1 protein present in the cells, Western blot was performed using cell lysates. No difference in the abundance of ABCA1 protein was found.

The major finding is that transfection of cells with sterol 27-hydroxylase stimulates cholesterol efflux. The stimulation of cholesterol efflux could be explained by two mechanisms. Without being limited by this hypothesis, firstly, appearance of hydroxycholesterol in the plasma membrane may change the properties of the membrane and facilitate the release of cholesterol and/or 27-hydroxycholesterol. Secondly, since 27-hydroxycholesterol is a ligand of the LXR receptor which regulates a number of genes involved in cholesterol homeostasis, in particular ABCA1 (Fu, X etal (2001)), 27-hydroxycholesterol may enhance cholesterol efflux via effects on these genes. Several of these findings point to the likelihood of the first mechanism. Firstly, there was no induction of ABCA1 in transfected cells. Secondly, the effect of transfection was more pronounced with whole plasma than with lipid free apoA-1, the opposite would be expected if the enhanced efflux resulted from stimulation of the expression of ABCA1. Furthermore transfection mainly stimulated the “rapid” phase of the efflux, i.e. release of cholesterol already present in the plasma membrane (Gaus, K etal (2001)) making involvement of pathways required for mobilization of intracellular cholesterol unlikely. Transfection of cells with CYP27 resulted in the production of 27-hydroxycholesterol when sterols were labeled metabolically, but not when the cholesterol pool was labeled with [³H] cholesterol. This may be due to the inability of exogenous cholesterol to access the mitochondrial enzyme (Cali, J. J., and Russell, D. W. (1991) J. Biol. Chem. 266, 7774-7778). Even with metabolic labeling the proportion of newly synthesized 27-hydroxycholesterol was low and increased efflux was found to be mainly if not entirely due to stimulation of the efflux of non-oxidized cholesterol.

### Example 2

### Expression of Caveolin-1 Enhances Cholesterol Efflux in Hepatic Cells

(a) Cells

HepG2 cells were cultured in DMEM, 10% fetal bovine serum, 100 μg/ml penicillin/streptomycin and 2 mM glutamine. The cells were seeded at a density of 1×10⁶ cells per well in a 6-well tissue culture plate and for 24 hours until the cells are 60-80% confluent.

To obtain stable transfectants, HepG2 cells were transfected with pIRES2-EGFP/caveolin-1 (Clontech) plasmid, using LipofectAMINE PLUS Reagent (Invitrogen) in serum-free medium for 5 hours at 37° C. according to manufacturer recommendations. Transfection medium was removed and fresh complete growth medium was added. Twenty-four hours post transfection, cells in one well were split into a 9-cm dish in medium containing 500 μg/ml G418 and cultured for 3-4 days until G-418-resistant colonies were clearly evident. Individual colonies were picked into 24 well plates to continue incubation with G418 selection medium. Individual colonies were evaluated for caveolin expression and independent monoclonal line was used for all experiments.

(b) Electron Microscopy

Cells were processed for embedding in resin and sectioned for electron microscopy.

(c) Confocal Microscopy

Cells were grown on sterile plastic cover slips to approximately 60% confluence. Cells were fixed in acetone for 20 min, washed with PBS and incubated for 1 h with anti-caveolin-1 antibody (Transduction Laboratories, KY). Cells were then washed again and incubated in the dark for 1 h with Texas Red labelled anti rabbit IgG. After mounting onto glass slides cells were observed using Zeiss META confocal microscope.

(d) Cholesterol Acceptors

Blood from healthy normolipidemic (plasma total cholesterol values ranging from 3.4 to 5.0 mmol/L) volunteers was collected in saline containing streptokinase (Sigma, final concentration 150 units/ml) and plasma was isolated by centrifugation for 15 min at 1500 g at 4° C. Plasma samples were not pooled, but rather used individually. Apolipoprotein A-I was isolated as described in Morrison, J. R., Fidge, N. H., and Grego, B. (1990) Anal. Biochem. 186, 145-152.

(e) Cholesterol and Phospholipid Efflux

HepG2 or HepG2/cav cells were grown to 80% confluence prior to experiments. The cultures were 100% confluent by the time of incubation with cholesterol acceptors. To label cellular cholesterol, cultures were incubated in serum-containing medium with [1α,2α(n)-³H]cholesterol (Amersham Pharmacia Biotech (APB), specific radioactivity 1.81 TBq/mmol, final radioactivity 0.2 MBq/ml) for 48 h in a CO₂ incubator. To label cellular phospholipids cells were incubated with [methyl-3H]choline (APB, specific radioactivity 3.11 TBq/mmol, final radioactivity 37 MBq/ml) for 24 h in a CO₂ incubator. After labelling, cells were washed six times with PBS and further incubated for 18 h in serum-free medium. For metabolic labeling of cellular cholesterol cells were incubated in serum-free Leibovitz L-15 medium with [³H]acetate (ICN, specific radioactivity 95.2 GBq/mmol; final radioactivity 7.4 MBq/ml) for 3 h at 15° C. Cells were washed and further incubated for 2 h at 37° C. in serum-free medium containing either lipid-free apoA-1 (final concentration 50 μg/ml), or cyclodextrin (final concentration 200 μg/ml) or the indicated concentrations of human plasma. The medium was then collected, centrifuged for 15 min at 4° C. at 30,000 g to remove cellular debris and the supernatant counted or used for further analysis. Cells were harvested using a cell scraper, dispensed in 0.5 ml distilled water and aliquots were counted or used for further analysis. Phospholipids and metabolically labeled cholesterol were isolated from aliquots of cells and medium by TLC as described in Sviridov, D., and Fidge, N. (1995) J. Lipid Res. 36, 1887-1896 and Sviridov, D., Pyle, L., and Fidge, N. (1996) J. Biol. Chem. 271, 33277-33283. Cholesterol and phospholipid efflux was expressed as a percentage of labeled cholesterol or phospholipid transferred from cells to the medium.

(f) Cholesterol Trafficking

To label the entire cellular cholesterol pool, cells were incubated in a serum-containing medium with [³H]cholesterol (final radioactivity 0.2 MBq/ml) for 48 h at 37° C. in a CO₂ incubator. After washing cells were further incubated in serum-free medium for 18 h at 37° C. in a CO₂ incubator. Cells were cooled on ice, [¹⁴C] acetate (ICN, final radioactivity 7.4 MBq/ml) was added and cells were further incubated in Leibovitz L-15 medium for 3 h at 15° C. Under these conditions intracellular cholesterol trafficking is blocked while cholesterol biosynthesis proceeds as described in Kaplan, M. R., and Simoni, R. D. (1985) J. Cell Biol. 101, 446-453 and Urbani, L., and Simoni, R. D. (1990) J. Biol. Chem. 265, 1919-1923. At the end of the incubation cells were quickly warmed and incubated for 20 min at 37° C. to allow a portion of the newly synthesized cholesterol to be transferred to the plasma membrane. The cells were cooled on ice, washed 3 times with the ice-cold phosphate buffered saline (PBS). Cholesterol oxidaze (Roche) was added to the cells at a final concentration of 1 unit/ml and flasks were incubated for 3 h at 4° C. Under these conditions only cholesterol in plasma membrane cholesterol-rich domains is oxidized forming cholestenone, and the reaction is carried out to completion. Lipids were extracted from cells and analyzed by TLC. “No oxidaze” and “no warm up” controls were included in each experiment. The amount of oxidazable cholesterol was calculated as the amount of [¹⁴C] or [³H]oxysterol as a fraction of total non-oxidized [³H]cholesterol in the sample (entire cholesterol pool) thus correcting for losses during lipid analysis. “No oxidaze” control, which was 10% of “oxidized” samples, was subtracted from the experimental values.

(g) Cholesterol Biosynthesis and Esterification

To assess cholesterol biosynthesis cells were incubated in serum-free medium with [³H]acetate final radioactivity 7.4 MBq/ml) for 20 min at 37° C. in a CO₂ incubator. To assess cholesterol esterification cells were incubated for 2 h at 37° C. with [¹⁴C]oleic acid (APB, specific radioactivity 2.22 GBq/mmol; final radioactivity 0.185 MBq/ml) complexed to BSA (Sigma, essentially fatty acid free). Cells were washed and lipids were extracted and analyzed by TLC as described in Sviridov, D., and Fidge, N. (1995) J. Lipid Res. 36, 1887-1896. Spots of cholesterol and cholesteryl oleate were identified by standards (Sigma), scraped and counted in a β-counter.

(h) Reverse Transcriptase PCR

Total RNA was extracted from CHOP cells following a modification of the guanidinium thiocyanate method. The RNA concentration was determined by measuring the absorption at 260 nm. Reverse transcription was carried out in 20 μl containing 20 mM Tris-HCl (pH 8.4), 50 mM KCl, 5 mM MgCl₂, 10 mM DTT, 40 U RNAse inhibitor and 50 U Superscript II reverse transcriptase (Invitrogen). PCR was performed in a total volume of 50 μl containing 10 mM Tris-HCl (pH 8.4), 50 mM KCl, 1.5 mM (GAPDH) or 5 mM (CYP27 μl) MgCl₂, 200 μM dNTPS, 100 ng of the appropriate forward and reverse primer (Caveolin 5′ primer GAGGGACATCTCTACACCGTTC; Caveolin 3′ primer ACTGAATCTCAA TCAGGAAGCTCT) 2 μl of reverse transcribed cDNA, 1 U Taq polymerase (Invitrogen). The reaction was amplified with a DNA thermal cycler (Perkin Elmer) for 35 cycles. The amplification profile involved denaturation at 94° C. for 15 seconds, primer annealing at 55° C. for 30 seconds, and elongation at 72° C. for one minute. 10 μl of each PCR reaction was mixed with 2 μl of six fold concentrated loading buffer and loaded on a 1% agarose gel containing ethidium bromide. Electrophoresis was carried out with a constant voltage for 1 h. The sequences of the fragments amplified by PCR were confirmed by DNA sequencing.

Amount of ABCA1 mRNA in HepG2 and HepG2/cav cells was quantitated by Real Time RT-PCR according to Su et al (31). Primers were: forward: 5′ TCCTCTCCCAGAGCAAAAAGC 3′; reverse: 5′ GTCCTTGGC AAAGTTCACAAATACT 3′; probe: 5′ ACTCCACATAGAAGACTACT 3′. 18S rRNA was used as an internal control and the results were expressed as ΔCt (difference between Ct values for ABCA1 and 18S RNAs)

(i) Western Blotting

Cells were lysed in RIPA buffer and proteins were separated on a 7.5% (ABCA1) or 10% (caveolin) SDS-polyacrylamide gel followed by immunoblotting, using either rabbit anti-ABCA1 serum (risen against recombinant fragment of ABCA1(1311-1450) or anti caveolin-1 antibody (Transduction Laboratories). Bands were visualized by chemiluminescence development and quantitated by densitometry.

Results

(a) Stable Transfection of HepG2 Cells with Caveolin-1

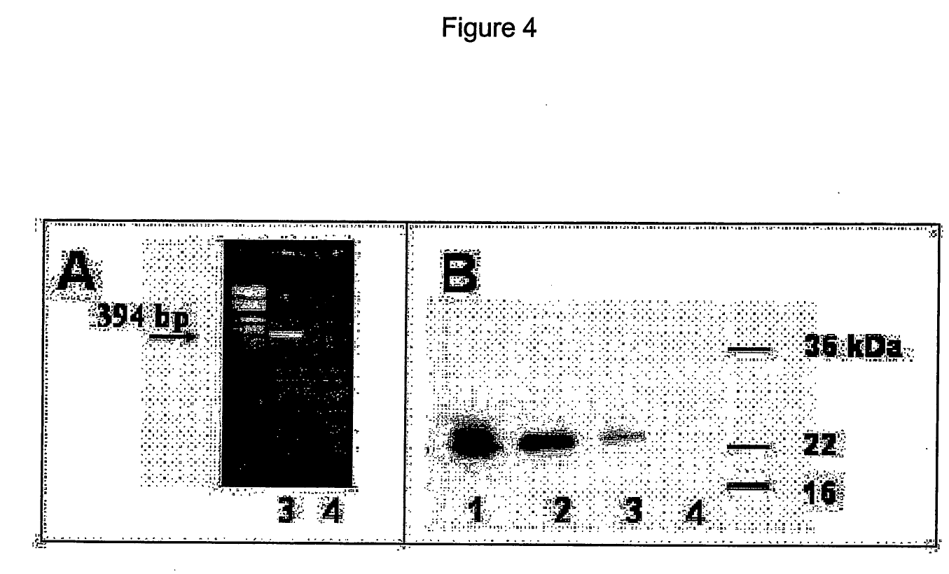

No expression of caveolin-1 mRNA or presence of caveolin was detected in non-transfected HepG2 cells (FIG. 4, A,B, line 4). When cell line of HepG2 stably transfected with caveolin-1 (HepG2/cav) was generated, a strong signal corresponding to caveolin mRNA was detected by RT PCR. (FIG. 4 A, line 3). Expression of caveolin was also analyzed by Western blot by using specific anti-caveolin antibodies. A single band migrating at the same position as caveolin from endothelial cells was detected in HepG2/cav cells (FIG. 4 B, lines 1-3). The plasmid used for transfection contained GFP enabling evaluation of the efficiency of the transfection. After final cloning 90% of cells were expressing GFP.

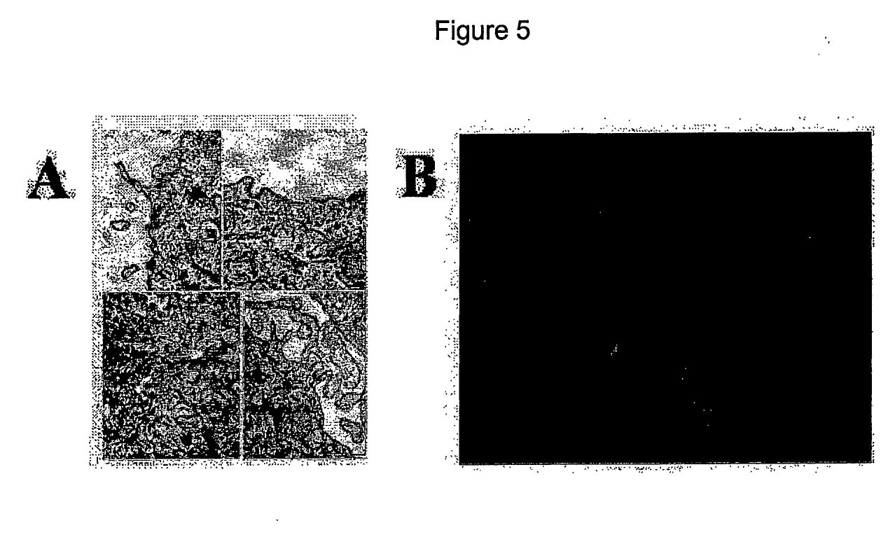

The caveolin-1 transfected cells were also examined by electron microscopy. Surface-connected pits with the typical morphology of caveolae and approximately 65 nm in size were observed in the transfected cells but not in control cells (FIG. 5 A). However, caveolae abundance was low with on average less than one caveolar profile per cell profile. Cells were also treated with anti-caveolin antibody and studied using confocal electron microscopy. HepG2/cav cells were stained with anti-caveolin antibody (FIG. 5 B) while control HepG2 cells showed no staining. Two patterns of staining were found in HepG2/cav cells. Approximately half of cells have majority of caveolin on their plasma membrane with only background staining in cytoplasm. The remaining half have caveolin distributed between perinuclear space and plasma membrane. This is consistent with caveolin present in plasma membrane caveolae and lipid bodies.

(b) Cholesterol Efflux

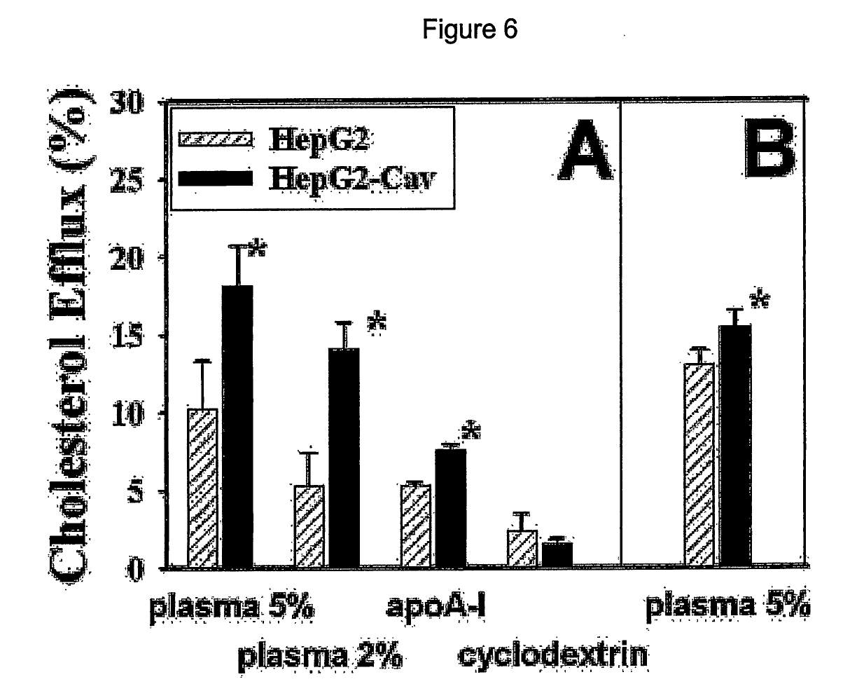

To evaluate the effect of caveolin expression on cholesterol efflux cellular cholesterol was labeled with [³H]cholesterol and cells were incubated with human plasma, lipid-free apoA-1, cyclodextrin and medium alone. Comparing to HepG2 cells the efflux from HepG2/cav cells was 80%, 280% and 45% higher (p<0.01 for all) when respectively 5% plasma, 2% plasma or 50 μg/ml of apoA-1 were used as an acceptor (FIG. 6 A). There was no difference in cholesterol efflux from two cell types to cyclodextrin (200 μg/ml), a non-specific cholesterol acceptor. When cellular cholesterol was metabolically labeled with [³H]acetate, the efflux from HepG2/cav cells to 5% plasma was 20% higher than from HepG2 cells (p<0.01) (FIG. 6B.).

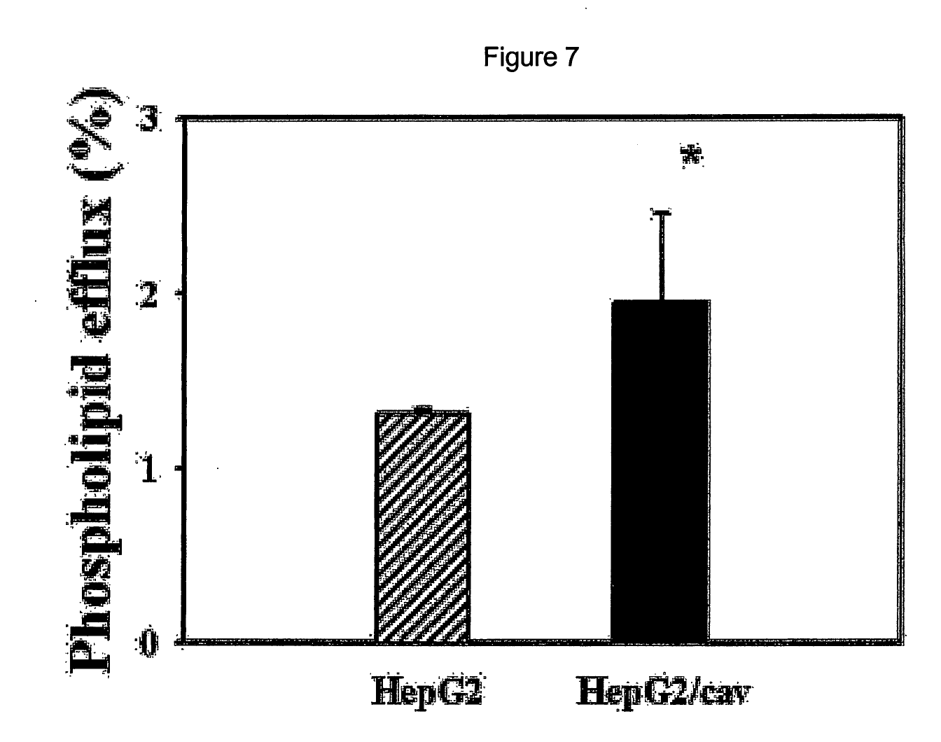

To analyze the efflux of phospholipids, they were metabolically labeled with [³H]choline and cells were incubated for 2 h with lipid-free apoA-1 (50 μg/ml). Phospholipid efflux from HepG2/cav cells was 50% higher compared to HepG2 cells (p<0.05) (FIG. 7).

(c) Intracellular Cholesterol Trafficking

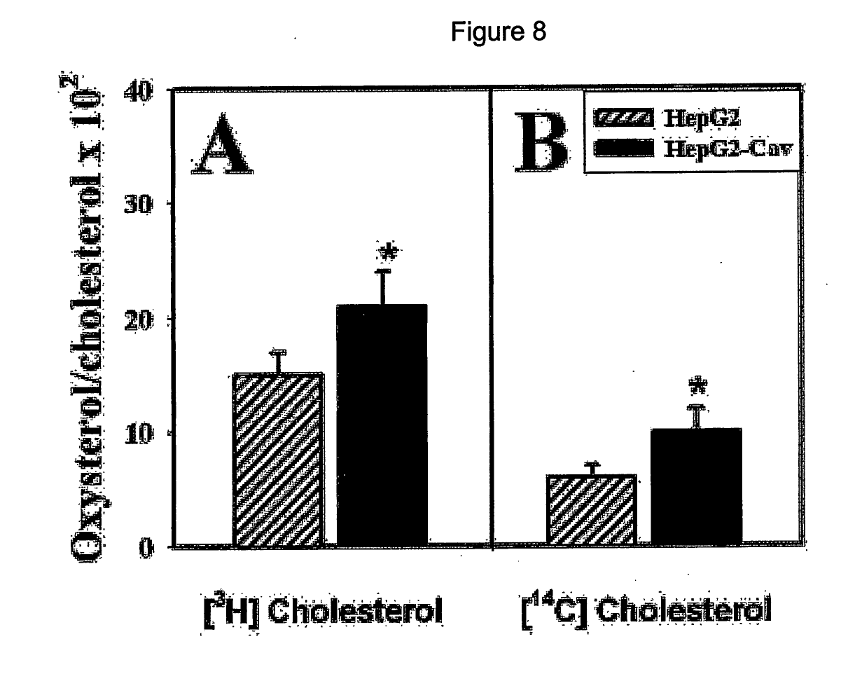

Cells were labelled with [³H]cholesterol allowing labeled cholesterol to equilibrate among all cellular pools. Cells were then treated with cholesterol oxidaze at conditions when only cholesterol in cholesterol rich-domains on cell surface (rafts and caveolae) is oxidized. The relative amount of cholesterol in cholesterol-rich domains of HepG2/cav cells was 40% higher compared to HepG2 cells (p<0.02) (FIG. 8 A). To assess the rate of cholesterol trafficking cells were incubated with [¹⁴C]acetate for 3 h at 15° C. Cells were then warmed up for 20 min at 37° C. to allow newly synthesized cholesterol to move to plasma membrane before treating cells with cholesterol oxidaze. The amount of newly synthesized [¹⁴C]cholesterol moved to the cholesterol rich domains of plasma membrane was 67% higher in HepG2/cav cells compared to HepG2 cells ((p<0.02) (FIG. 8B).

(d) Cholesterol Biosynthesis and Esterification

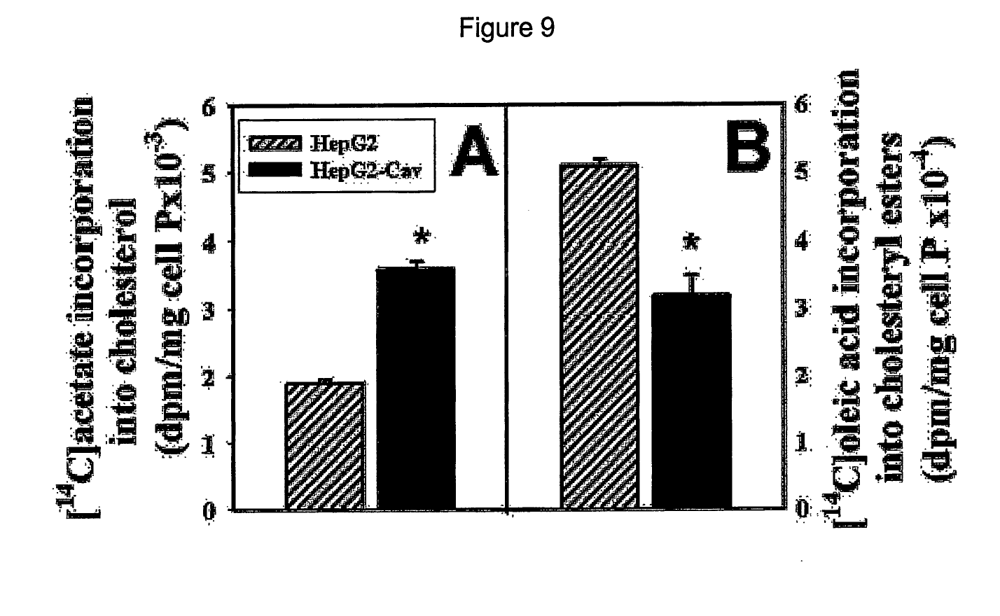

Cholesterol biosynthesis and cholesterol esterification in HepG2 and HepG2/cav cells were assessed as an indirect measure of changes in cellular cholesterol content. The rate of [3H]acetate incorporation into cholesterol in HepG2/cav cells was almost twice that of HepG2 cells (p<0.001) (FIG. 9A). In contrast, the rate of [¹⁴C]oleic acid incorporation into cholesteryl esters in HepG2/cav cells was 40% slower compared to HepG2 cells (p<0.001) (FIG. 9B).

(e) Cell Proliferation

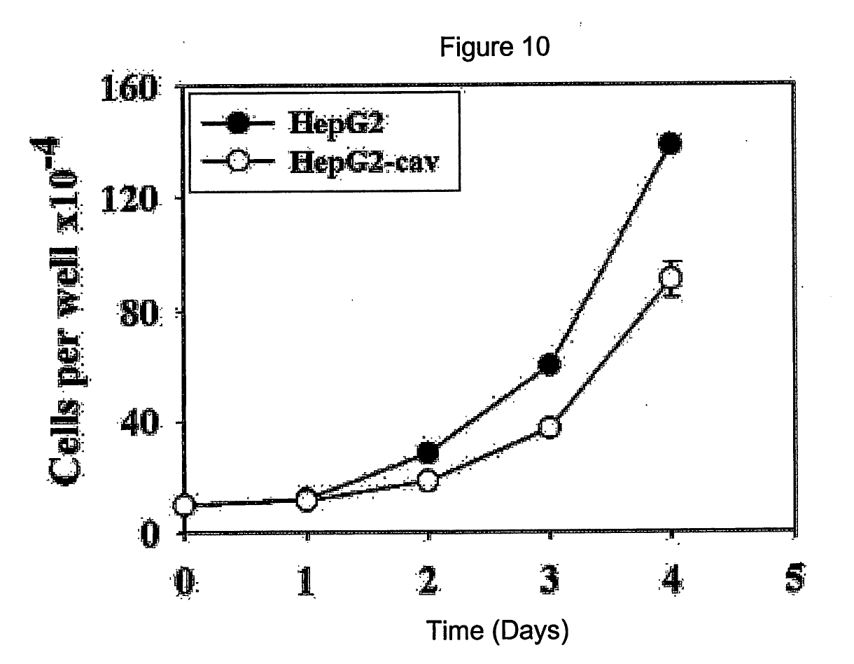

Cell cholesterol content is tightly connected to cell growth and increase in cholesterol efflux inhibits cell proliferation. When proliferation rate was analyzed in HepG2 and HepG2/cav cell cultures seeded at the same density, the rate was significantly slower in HepG2/cav cells. Fifty one percent fewer cells was found in HepG2/cav cultures then in HepG2 cultures after four days of culturing, when the cultures were approaching confluent state (p<0.001) (FIG. 10).

(f) Secretion of apoA-I

HepG2 cells being hepatic cell line produce and secretes VLDL and apoA-I which may contribute to release of cholesterol from cells. We found that HepG2 cells produce substantial amounts of apoA-1, most of produced apoA-I is secreted to the medium (Table 1). However the amount of apoA-I secreted by both cell types during 2 h incubation was about 10% of the amount of exogenous apoA-1 added (Table 1). Interestingly, the amount of apoA-I synthesized and secreted by HepG2/cav cells was 3 times higher than by untransfected cells (Table 1). Thus, the observed stimulation of cholesterol efflux is not related to apoA-I secretion by the cells.

TABLE 1
Synthesis and secretion of apoA-I by HepG2 and HepG2/cav cells
Amount of apoA-I in the cells and medium after 2 h incubation was
analysed by competitive ELISA.

| | | Secreted apoA-I | |
|---|---|---|---|
| | Intracellular apoA-I | μg/mg of cell | |
| Cells | μg/mg of cell protein | protein | μg/ml |
| HepG2 | 0.25 ± 0.01 | 2.8 ± 0.2 | 5.4 ± 0.3 |
| HepG2/cav | 0.21 ± 0.12 | 0.8 ± 0.1** | 2.4 ± 1.1* |

Background efflux (i.e. efflux in the absence of any exogenous acceptors) was below 10% and was similar for two cell types. This discounted a possibility that cholesterol released with secreted VLDL and apoA-I contributed significantly to the observed differences in cholesterol efflux.
(g) Expression of ABCA1

It was demonstrated by Orso et al that mutations of ABCA1 affect caveolin-1 expression and processing In view of possible link between expression of caveolin and ABCA1 we investigated the effect of stable transfection with caveolin on ABCA1 expression. However, when analyzed with Real Time RT-PCR, no difference in abundance of ABCA1 mRNA between two cell types was found Neither there was any difference in abundance of ABCA1 protein.

Accordingly, this study shows that stable transfection of HepG2 cells, a human hepatocyte-derived cell line, with caveolin-1 enhances cholesterol efflux.

### Example 3

### Expression of Sterol 27-hydroylase (CYP27A1) Enhances Cholesterol Efflux in Macrophages

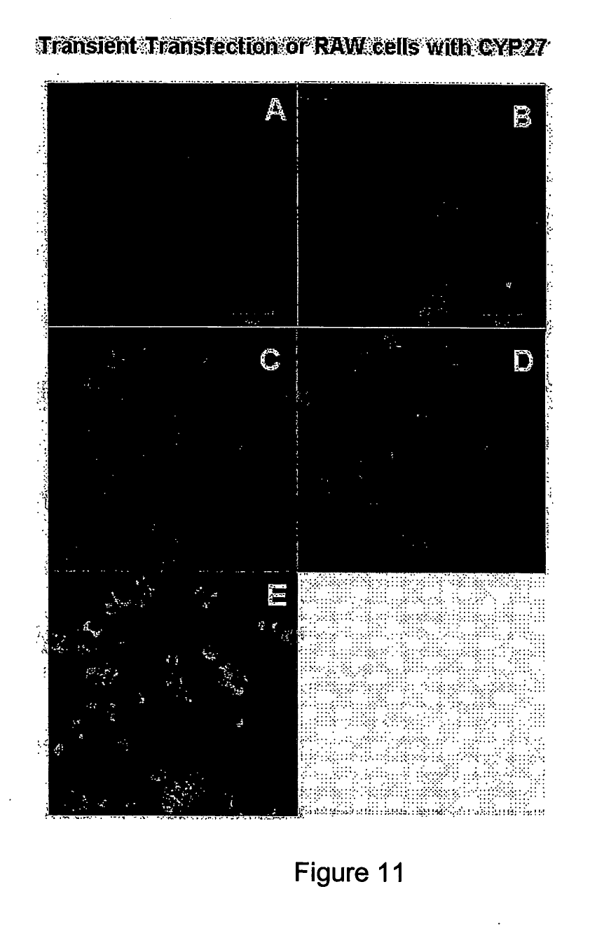

Macrophages were cultured and transfected the same as for the CHOP cells in Example 1 except that after transfection, 0.5 μM 5-azacytidine was included in all further incubations. Transfection of macrophages with high efficiency was always a difficult task. No more than 20% of cells were transfected. However it was found that in most cells the CMV promoter was rapidly silenced by methylation. Accordingly, a demethylation agent, 5-azacytidine, was added to the cells which then expressed CYP27. Panels A and B in FIG. 11 are actually transfected cells, but not treated with 5-azacytidine.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

Finally it is to be understood that various other modifications and/or alterations may be made without departing from the spirit of the present invention as outlined herein.

1. A method of modulating cholesterol efflux in a cell, said method comprising
modulating expression and/or activity of sterol 27-hydroxylase (CYP27) and/or caveolin-1 or equivalent thereof in the cell.

2. A method according to claim 1 wherein the cell is selected from the group including hepatic cells or hepatocytes, macrophages, endothelial cells, smooth muscle cells and other cells of the vessel wall and stem cells.

3. A method according to claim 2 wherein the expression and/or activity of CYP27 and/or caveolin-1 or equivalent thereof is modulated in a cell deriving from a vessel wall associated with an atherosclerotic plaque.

4-8. (canceled)

9. A method according to claim 1 wherein the cholesterol effluxed is substantially non-oxidised cholesterol.

10. A method according to claim 1 wherein the cell is transfected with an operably linked CYP27 and/or caveolin-1 gene or equivalent thereof to modulate expression and/or activity of CYP27 and/or caveolin-1 in the cell.

11. A method according to claim 1 wherein the cell is further treated with a demethylating agent.

12. A method according to claim 11 wherein the demethylating agent is 5-azacytidine.

13. A method of increasing cholesterol efflux in a cell, said method comprising increasing expression and/or activity of sterol 27-hydroxylase (CYP27) and/or caveolin-1 or equivalent thereof in the cell.

14. A method according to claim 13 wherein the expression of the gene encoding CYP27 and/or caveolin-1 or equivalent thereof is increased.

15. A method according to claim 13 wherein the activity of CYP27 and/or caveolin-1 or equivalent thereof is increased.

16. A method according to claim 13 wherein the cell is transfected with a gene encoding CYP27 and/or caveolin-1 or equivalent thereof and expression of the gene is increased to increase cholesterol efflux.

17. A method according to claim 13 wherein the cell is further treated with a demethylating agent.

18. A method according to claim 17 wherein the demethylating agent is 5-azacytidine.

19. A method according to claim 14 wherein the gene is induced to increase expression and/or activity of CYP27 and/or caveolin-1 or equivalent thereof.

20. A method according to claim 13 wherein the cell is selected from the group including hepatic cells or hepatocytes, macrophages, endothelial cells, smooth muscle cells and other cells of the vessel wall and stem cells.

21. A method according to claim 20 wherein the expression and/or activity of CYP27 and/or caveolin-1 or equivalent thereof is modulated in a cell deriving from a vessel wall associated with an atherosclerotic plaque.

22-26. (canceled)

27. A method according to claim 13 wherein the cholesterol effluxed is substantially non-oxidised cholesterol.

28. A cell having modulated cholesterol efflux, said cell comprising modulated expression and/or activity of CYP27 and/or caveolin-1 or equivalent thereof.

29. A cell according to claim 28 wherein the cell is transfected with a gene encoding CYP27 and/or caveolin-1 or equivalent thereof.

30. A cell according to claim 28 selected from the group including hepatic cells or hepatocytes, macrophages, endothelial cells, smooth muscle cells, cells of the vessel wall and stem cells.

31-33. (canceled)

34. A cell according to claim 28 having increased cholesterol efflux, said cell comprising increased expression and/or activity of CYP27 and/or caveolin-1 or equivalent thereof.

35. A method of treating a cholesterol related condition in a patient by modulating cholesterol efflux from a cell of the patient, said method comprising:
modulating expression and/or activity of CYP27 and/or caveolin-1 or equivalent thereof in the cell.

36. A method according to claim 35 by modulating cholesterol efflux in a cell in the patient, said method comprising:
introducing a gene construct to modulate expression and/or activity of CYP27 and/or caveolin-1 or equivalent thereof in the cell of the patient.

37. A method according to claim 36 wherein the gene construct includes a gene encoding CYP27 and/or a CYP27 regulator and/or caveolin-1 and/or a caveolin-1 regulator.

38. A method according to claim 35 wherein the expression and/or activity of CYP27 and/or caveolin-1 is increased.

39. A method of treating a cholesterol related condition in a patient by modulating cholesterol efflux in the patient, said method comprising:
introducing a modulated cell to the patient, wherein said cell comprises modulated expression and/or activity of CYP27 and/or caveolin-1 or equivalent thereof.

40. A method according to claim 39 wherein the modulated cell is transfected with a gene encoding CYP27 and/or caveolin-1 or equivalent thereof.

41. A method according to claim 39 wherein the expression and/or activity of CYP27 and/or caveolin-1 or equivalent thereof in the modulated cells is increased.

42. A method according to claim 39 wherein the cells are selected from the group including hepatic cells or hepatocytes, macrophages, endothelial cells, smooth muscle cells and other cells of the vessel wall and stem cells.

43-47. (canceled)

48. A method according to claim 39 wherein the cells are introduced into a region of disease associated with cholesterol accumulation.

49. A method according to claim 48 wherein the cells are introduced to vessel cells which line the vessel.

50. A method according to claim 39 wherein the cholesterol related condition is selected from the group including myocardial infarction, atherosclerosis, stroke, hypoalphalipoproteinaemia or peripheral vascular disease.

51. A method according to claim 50 wherein the cholesterol related condition is atherosclerosis.

52. A method of identifying a compound which modulates cholesterol efflux in a cell, said method comprising:
contacting the compound to the cell;
detecting a change in expression and/or activity of CYP27 and/or caveolin-1 or equivalent thereof in the cell relative to a cell which has not been contacted with the compound.

53. A method according to claim 52 wherein the cell is selected from the group including endothelial cells, smooth muscle cells and vessel cells, hepatic cells or hepatocytes, macrophages, and stem cells.

54. (canceled)

55. A method according to claim 53 wherein the cell is a hepatocyte transfected with caveolin-1.

56. A method according to claim 55 wherein the cell is a HepG2 cell.

57. A composition comprising:
a compound identified by the method of claim 52, which compound is present in an amount effective to treat a cholesterol-related condition.

58. (canceled)

59. A method according to claim 35 wherein the cells are selected from the group including hepatic cells or hepatocytes, macrophages, endothelial cells, smooth muscle cells and other cells of the vessel wall, and stem cells.
